(19) United States
(12) Patent Application Publication
(10) Pub. No.: US 2025/0134924 A1
(43) Pub. Date: May 1, 2025

(54) Composition for Prevention and Treatment of Bacterial and Viral Infections and Inflammation

(71) Applicant: David A. Cuddeback, Kirkwood, NY (US)

(72) Inventors: David A. Cuddeback, Kirkwood, NY (US); Thomas J. Lynch, Elverson, PA (US)

(73) Assignee: David A. Cuddeback, Kirkwood, NY (US)

(21) Appl. No.: 19/011,109

(22) Filed: Jan. 6, 2025

Related U.S. Application Data
(63) Continuation-in-part of application No. 18/410,512, filed on Jan. 11, 2024.
Application No. 18/410,512 is a division of application No. 18/094,541, filed on Jan. 9, 2023, now Pat. No. 11,896,611.
Application No. 18/094,541 is a division of application No. 17/682,300, filed on Feb. 28, 2022, now Pat. No. 12,186,341.
Application No. 17/682,300 is a continuation-in-part of application No. 16/848,393, filed on Apr. 14, 2020, now abandoned.
(63) Continuation-in-part of application No. 17/682,300, filed on Feb. 28, 2022, now Pat. No. 12,186,341.

(51) Int. Cl. A61K 33/30; A61K 9/00; A61K 9/48; A61K 31/05; A61K 31/12; A61K 31/198; A61K 31/202; A61K 31/352; A61K 31/355; A61K 31/375; A61K 31/4525; A61K 36/15; A61K 36/185; A61K 36/233; A61K 36/28; A61K 36/324; A61K 36/328; A61K 36/537; A61K 36/74; A61K 36/82

(52) U.S. Cl. CPC A61K 33/30; A61K 9/0053; A61K 9/48; A61K 31/05; A61K 31/12; A61K 31/198; A61K 31/202; A61K 31/352; A61K 31/355; A61K 31/375; A61K 31/4525; A61K 36/15; A61K 36/185; A61K 36/233; A61K 36/28; A61K 36/324; A61K 36/328; A61K 36/537; A61K 36/74; A61K 36/82

(57) ABSTRACT

Compositions and methods for protecting against a wide spectrum of viral and bacterial infections, including Covid-19, and for treating established infection and infectious inflammation are described. The composition includes a novel combination of vitamins, minerals, nutraceuticals, and phytochemicals, which may be compounded as a pill, tablet, powder, capsule or liquid to be taken orally or via other administration routs one or more times per day, to serve as immune boosters, antibacterial agents, and antiviral agents along with providing anti-inflammatory effects in humans and animals.

### CROSS-REFERENCE TO RELATED APPLICATIONS

This application claims priority from and is a continuation-in-part of: (i) U.S. nonprovisional patent application Ser. No. 18/410,512 filed Jan. 11, 2024, which is a divisional of U.S. nonprovisional patent application Ser. No. 18/094,541 (now U.S. Pat. No. 11,896,611) filed on Jan. 9, 2023, which is a divisional of U.S. nonprovisional patent application Ser. No. 17/682,300 filed on Feb. 28, 2022, which is a continuation-in-part of U.S. nonprovisional patent application Ser. No. 16/848,393 filed on Apr. 14, 2020; and (ii) U.S. nonprovisional application Ser. No. 17/682,300 filed on Feb. 28, 2022, which is a continuation-in-part of U.S. nonprovisional patent application Ser. No. 16/848,393 filed on Apr. 14, 2020. All of the foregoing applications and issued patents are incorporated by reference herein in their entireties.

### FIELD OF THE INVENTION

The invention relates to compositions and methods for preventing and for treating and reducing infections and tissue inflammation caused by bacteria and viruses in humans and animals. More particularly, the invention relates to compositions and methods that include multimodal prophylaxis against and treatment of viral and bacterial infections and viral and bacterial induced inflammations, especially in humans.

### BACKGROUND

Several categories of inflammatory diseases exist. Two of them are: (i) sero-negative spondyloarthropathies (e.g., osteoarthritis, gouty arthritis, pseudogout, reactive arthritis, Lyme disease, psoriatic arthritis, and septic arthritis); and (ii) sero-positive spondyloarthropathies, also known as autoimmune diseases (e.g., rheumatoid arthritis, lupus, systemic sclerosis, Sjogren's syndrome, polymyalgia rheumatica, polymyositis/dermatomysitis, and sarcoidosis). Fibromyalgia is not an autoimmune disease per se, but the pain it causes is correlated with inflammatory cytokines (see below).

Across these two categories, the most frequently cited inflammatory cytokines involved in the overall painful and tissue-destructive inflammations are IL-1β, IL-6, IL-8, IFN-γ and TNFα. Generally, inflammatory diseases and conditions have been treated using steroids, which are the most powerful anti-inflammatoires. Steroids are used as the anti-inflammatory of last resort in severe autoimmune disease flare-ups (e.g., rheumatoid arthritis and lupus) and in treating the severe pneumonia caused by COVID. However, they have serious side effects, especially when used long term. Since steroids create serious, new clinical problems, any drug that can be used in their stead is said to be steroid-sparing. Avoiding steroids when possible is an established clinical directive.

Nutraceuticals are one such possible non-steroidal approach to the treatment of inflammatory diseases and conditions. Nutraceuticals include: (a) dietary supplements, such as vitamins, minerals, herbs, amino acids, enzymes, organ tissues, and metabolites; and (b) functional foods, which have been defined in a position statement by the Academy of Nutrition and Dietetics: Functional Foods. Kristi Crowe; Coni Francis, J Acad Nutr Diet. 2013; 113:1096-1103, as “whole foods along with fortified, enriched, or enhanced foods that have a potentially beneficial effect on health when consumed as part of a varied diet on a regular basis at effective levels based on significant standards of evidence.” Examples of functional foods would include “the antioxidant vitamins in orange juice, isoflavones in soy-based foods, and prebiotics and probiotics in yogurt.” Id. In all such instances, the antioxidant or other functional ingredient is contained within an intact, whole food.

Phytonutrients, defined by the NIH/National Cancer Institute are “organic components of plants thought to promote human health. Fruits, vegetables, grains, legumes, nuts and teas are rich sources of phytonutrients. Unlike the traditional nutrients (protein, fat, carbohydrates, vitamins, and minerals), phytonutrients are not ‘essential’ for life, so some people prefer the term Phytochemical.”

Phytochemicals, within the parent plant, help the plant resist microbial attack by fungi, bacteria and viruses. Phytochemicals are further defined by Liviuta Budisan et al; Int J Mol Scvi. 2017 June; 18(6): 1178, as “natural compounds synthesized as secondary metabolites in plants, representing an important source of molecules with a wide range of therapeutic applications.” Budisan et al. explains further that “these natural agents are important regulators of key pathological processes/conditions, including cancer, as these natural agents are able to modulate the expression of coding and non-coding transcripts with an oncogenic or tumor suppressor role.”

Phytochemicals are also known to have multiple antiviral effects in vitro and multiple anti-inflammatory effects in vitro and in vivo.

For phytochemicals to be most effective in their use, they must be extracted in concentrated form from the parent plant. Chemical classifications of the family of phytochemicals are all slightly different.

Polyphenols are phytochemicals found largely in fruits, vegetables, tea, coffee, chocolates, legumes, cereals, and beverages. Over 8,000 polyphenols have been identified in nature and their main function is antioxidant and other significant health benefits.

Many effective drugs have come originally from phytochemical sources. A selected few examples follow: Willow bark was used more than 3,500 years ago as an analgesic and antipyretic, although those terms were not used at the time. About 1897, Felix Hoffman discovered its active component to be the phytochemical salicin, which inspired his synthesis of salicylic acid (aspirin). Atropine first came from the belladonna plant (i.e., nightshade) and is now used to decrease respiratory secretions during surgery, to treat poisoning by certain cholinergic nerve agents and as the first line treatment for symptomatic bradycardia in the emergency department. Paclitaxel was isolated from the English Yew tree in 1971 and has since been used to treat ovarian, breast, and non-small cell lung cancer. In 1928, Alexander Fleming discovered the first antibiotic, penicillin, in mold of the genus Penicillium.

There is renewed interest in phytochemicals because of more recent research that has demonstrated antiviral and antibacterial efficacy of various phytochemicals in numerous in vitro experiments and especially now because of the prophylactic and therapeutic quandary presented by the SARS COV-2 pandemic. The COVID-19 oral antiviral pills recently introduced by Pfizer, Paxlovid (ritonavir), and by Merck, Lagevrio (molnupiravir), were first discovered in in vitro research, then confirmed in in vivo (human) clinical trials. It is a common research pattern that positive results in vitro often become positive results in vivo (i.e., in humans and animals).

Phytochemicals are not food, vitamins or minerals per se. Rather, they are plant chemicals with multiple, evolved non-nourishment purposes, particularly plant defense. Of note, their molecular structures can bear resemblance to laboratory chemicals invented for human defense.

Before modern pharmaceuticals, mankind depended on natural substances for medical purposes which included, but were not limited to, relieving pain, enhancing sexual and other performance, enhancing sleep, enhancing bowel function, healing wounds, decreasing respiratory difficulty, stopping blood loss, halting the progress of illness and augmenting general wellbeing. Such natural substances included, but were not limited to, vegetables, herbs, roots, flowers, tree barks and certain animal organs and tissues. A key target of the use of natural substances was abatement of the combination of pain, swelling and redness in any area of the body, a combination which by the 16th century came to be known as inflammation. At that time and before, inflammation was an important target for treatment with natural substances because it was well recognized that inflammation could progress to worse, morbidity or death. Although allergic or purely traumatic injury can also cause inflammation, in the 19th century it was discovered that bacterial infection was a chief and more dangerous cause. In the 20th century, knowledge of the causes of inflammation expanded to include viral and fungal infections.

In our time, natural substances have evolved to include isolated atoms or molecules known as minerals, isolated biomolecules known as vitamins, and purified extracts from any of the above mentioned, historical sources of natural substances. A large variety of such modern natural substances exists, which in modern pill, tablet, liquid, powder or capsule form are now known as dietary or health supplements, or nutraceuticals. Nutraceuticals and phytochemicals are currently used for essentially the same array of purposes as those throughout history, but now also for fortification of the immune system against primary acquisition of infection, i.e., for prophylaxis and for fortification of the immune system's ability to defeat infection once it is established.

Modern antibacterial and antiviral vaccines are generally effective against only the bacterial or viral pathogen from which they were pharmaceutically derived, not a range of bacteria or viruses. Modern (antibacterial) antibiotics for use after infection has already occurred are typically quite effective against some range of bacteria. Historically, however, antiviral medications for use after viral infection has already occurred, work against only a specific virus, have minimal and even questionable effectiveness, and also have severe time constraints on their uses.

A small variety of available supplements are targeted at overall fortification of the innate immune system, with the intent to prevent or mollify a variety of viral and bacterial infections such that no infection occurs, or that its symptoms and physiologic effects may be restricted to the minimal and the brief. These supplements go by such labels as Immune Boost, Immune Health and others, and many, if not most, are composed of a single active ingredient, and none claims both immune fortification and inclusion of a known anti-inflammatory substance.

Phytochemical extracts do not exist in nature. Further, any extract of a plant contains not one phytochemical but an array of phytochemicals. The above combination of vitamin C, thiamine, and hydrocortisone used for sepsis is referred to as a clinical cocktail. Clinical cocktails are little used in medicine. Instead, the tradition in Western medicine is one clinical drug for one clinical problem, which is not always successful. The purpose of a clinical cocktail is to overwhelm the clinical problem by causing multiple corrective effects at once.

A need exists for a novel and optimal combination of immune fortifying ingredients, each of which works by a different mechanism to prevent, mollify and treat a wide range of viral and bacterial infections (hereafter termed wide-spectrum anti-infectious), and one or more additional ingredients that are anti-inflammatory, in the event that some degree of infectious inflammation becomes established.

Since whole-food nutraceuticals are an inefficient and minimally effective source of immune-fortifying, antiviral and anti-inflammatory ingredients, a need also exists for a novel and unique combination of extracted phytochemicals whose wide-spectrum prophylactic effect against infection is compounded by its ability to treat already established infection and infectious inflammation, such that its overall prophylactic and therapeutic effects synergistically equal more than the sum of its parts, for example, as does the fortuitous combination of vitamin C, thiamine, and hydrocortisone in reversal of sepsis, a severely life-threatening condition that can follow routine infection with a virus or bacterium.

A further need exists for steroid sparing or non-steroidal compositions and methods for treating inflammation.

### SUMMARY

The invention relates to compositions and methods for protecting against a wide spectrum of viral and bacterial infections, including Covid-19, and for treating established infection and infectious inflammation caused by viruses and bacteria. The composition described herein include a novel combination of vitamins, minerals, nutraceuticals, and phytochemicals. The compositions may be produced as a pill, tablet, powder, capsule, nasal spray, inhalable form, suspension, a topically applied form, or liquid to be taken orally or via other administration routes one or more times per day. The compositions act as immune boosters, antibacterial agents, and antiviral agents along with providing anti-inflammatory effects in humans and animals.

To protect against viral infection, we propose a combination nutraceuticals that support the innate immune system (e.g., zinc, vitamin C, and vitamin E) plus an unnatural combination (cocktail) of antiviral phytochemical extracts derived from different plants, wherein each extract itself contains its own sub-array of antiviral and antibacterial phytochemicals. Representative literature references citing the in vitro antiviral and/or antibacterial efficacy of the phytochemical extracts contained in the compositions described herein include those for extracted fulvic acid, Radix bupleuri extract, Uncaria tomentosa extract, pomegranate extract, black elderberry extract, sage extract, and echinacea extract.

The invention utilizes a phytochemical cocktail approach to prevent or treat health-threatening inflammation caused by an established viral or bacterial infection. To protect against infectious inflammation, the invention includes a method and a composition having a combination of anti-inflammatory nutraceuticals (e.g., omega-3 fatty acid and alpha-lipoic acid) plus an unnatural combination (cocktail) of anti-inflammatory phytochemical extracts derived from different plants, wherein each extract itself contains its own sub-array of anti-inflammatory phytochemicals. Representative literature references citing the in vitro and/or in vivo anti-inflammatory efficacy of the phytochemical extracts contained in the compositions described herein include those for Boswellia serrata extract, maritime pine bark extract, Uncaria tomentosa extract (which also has antiviral and antibacterial effects), Commiphora myrrha extract, resveratrol, alpha-lipoic acid, curcumin extract with piperine, green tea extract, omega-3 fatty acids, and cannabidiol.

The nutraceuticals contained in the proposed antiviral cocktail in the compositions and methods described herein support numerous facets of the innate immune system, and the phytochemicals contained therein combat many or most of the mechanisms of viral infection and replication. The nutraceuticals and phytochemicals contained in the anti-inflammatory cocktail compositions described herein inhibit or block a key set of chemical mediators of inflammation (e.g., cytokines) that cause serious viral inflammation, and notably those that cause cytokine storm in COVID-19. These mediators include IL-1β, IL-6, NF-kappa B, leukotrienes, TNF-alpha and the enzyme, human leukocyte elastase, believed to cause lung tissue destruction in emphysema. among sero-negative spondyloarthropathies and sero-positive spondyloarthropathies that cause inflammation, IL-1β, IL-6, IL-8, IFN-γ and TNFα are the most frequently cited inflammatory cytokines involved in the overall painful and tissue-destructive inflammations. Both in vitro and in vivo research has shown that all of the above inflammatory cytokines are redundantly inhibited or blocked by the anti-inflammatoires described herein. In COVID, the production of these same inflammatory cytokines was so great, destructive, and often fatal that it was called cytokine storm.

In addition to the above anti-inflammatory influences, several of the anti-inflammatory compounds described herein also block nuclear factor kappa B (NFκB), which is a master nuclear transcription factor that when activated triggers the production of many of the above inflammatory cytokines. Further, some of the anti-inflammatory compounds described herein also block the production of the extremely inflammatory leukotrienes.

Taurine blocks or inhibits most of the same of mediators of inflammation including, for example, IL-1β, IL-6, IL-8, TNFα, and NFκB. Like many of the phytochemical anti-inflammatories included in the compositions described herein, taurine is also an antioxidant because of its ability to scavenge cell-toxic reactive oxygen species (ROS), i.e., the hydroxyl radical (OH), the hypochlorous radical (HOCl), superoxide (O₂⁻), hydrogen peroxide (H₂O₂), and singlet oxygen (1O₂).

In addition to cytokines, oxygen free radicals are also known to be key players in inflammatory processes. So too, inflammatory processes in the vascular endothelium cause endotheliitis in COVID-19, which enables thrombogenesis and which in turn creates multiple clots that cause stroke, heart attack, and renal failure. Another facet of the anti-inflammatory phytochemical cocktail compositions described herein, with their inherent subarrays of other phytochemicals, is that many of the phytochemicals contained therein also have antioxidant and/or antithrombotic effects.

Still another facet of the compositions and methods described herein is that desired effects, whether antiviral, antibacterial, anti-inflammatory, antioxidant, or antithrombotic, should be potentiated by the effects of each component compounding the effects of the others. This has been found for pomegranate rind extract and punicalagin when co-administered with zinc in its virucidal activity against both herpes simplex virus (HSV) and acyclovir-resistant HSV. Of the multiple, possible mutual potentiations among our phytochemicals, synergy between two or more is also possible, synergy in which the combined effect of two or more is significantly greater than the sum of their individual effects. Synergy has been found for the anti-inflammatory effect of the co-administration of Commiphora myrrha extract with Boswellia serrata extract.

A common feature of laboratory-invented antiviral medications (e.g., oseltamivir and zanamivir) used against influenza virus is that strains of influenza (e.g., H1N1 and H3N2) rapidly develop resistance to the medication and thereafter become ineffective in certain populations. A curiosity of several of our proposed anti-infective phytochemicals is that even after multiple passes in vitro, the viruses against which they were being tested failed to develop resistance to the phytochemicals, whereas the same number of in vitro passes caused significant viral resistance against laboratory-invented antiviral medications. There is as yet no explanation for this advantage of phytochemicals.

By and large, research has not tested the combination of even two co-administered phytochemicals for their anti-infectious or anti-inflammatory effects, let alone a cocktail of three or more. An unexpected result thereof, or perhaps a foregone conclusion, should be that since each of these phytochemical extracts has been shown to be effectively antiviral against one or more viruses in vitro, then a cocktail of all multiple, combined extracts should possess an even greater antiviral effect against any one virus and be effectively antiviral against a much wider range of different viruses.

A novel combination of vitamins, minerals and herbal or other natural, organic supplements (i.e., phytochemicals) for protection against a wide spectrum of viral and bacterial infections and for treatment of established infection and infectious inflammation is desirable. The compositions described herein containing phytochemical anti-inflammatoires is believed to improve quality of life by treating some of the above conditions and thereby to be steroid-sparing. The compositions described herein containing combinations of vitamins, minerals and phytochemical supplements can be produced as a tablet, a powder, a pill, a capsule, a nasal spray, an inhalable form, a suspension, a topically applied form, or a liquid for administration to a human or other animal via oral, parenteral, inhalation, topical application, or other administration routes.

Among the possible innate immune system-supporting vitamins to be used are vitamins C and E. The mineral to be used is zinc complexed with citrate, pyrithione, or the amino acid methionine, or other cofactor, in order to increase zinc's bioavailability.

Phytochemicals provided herein as immune boosters and/or antiviral agents to be combined as a composite in one vehicle include Echinacea purpura extract, extracted fulvic acid, black elderberry extract, sage extract, Cat's Claw extract (Uncaria tomentosa), pomegranate extract, Radix bupleuri extract (which contains saikosaponins) and cannabidiol (CBD).

The anti-inflammatory phytochemicals and nutraceuticals to be used are Boswellia serrata extract, maritime pine extract, Cat's Claw extract (Uncaria tomentosa), green tea extract, resveratrol, curcumin with piperine, alpha lipoic acid, Commiphora myrrha extract, cannabidiol (CBD) and the omega-3 fatty acids, DHA and EPA. Methods of using these compositions of vitamins, minerals, herbs and other organic substances and compositions are also disclosed herein. The amount of each individual supplement substance may be varied significantly.

An object of the invention is to provide compositions of phytochemical extracts and nutraceuticals to fortify the immune systems of humans and animals in a way that prevents a wide spectrum of known or new strains of viruses and bacteria from starting a new infection in the host.

Another object of the invention is to provide compositions of phytochemical extracts and nutraceuticals to fortify the immune systems of humans and animals in a way that permits nascent viral or bacterial infection, once established, to be treated and defeated before the infection can cause serious and/or widespread systemic symptoms and effects, or to minimize such symptoms to a life non-threatening level.

Another object of the invention is to provide compositions of phytochemical extracts and nutraceuticals that prevent or limit inflammation, especially in the lungs, in which virally or bacterially induced inflammation causes arteriolar and capillary leakage and resultant pulmonary edema, excess mucus secretion, possible severe bronchoconstriction and, in the worst case, acute respiratory distress syndrome (ARDS) with airway collapse, any of which phenomena can compromise pulmonary function to the point of fatal respiratory failure.

Another object of the invention is to provide compositions of anti-infective (i.e., antiviral and antibacterial) phytochemical extracts and nutraceuticals, already established to be safe for consumption that can be taken electively before anticipated viral exposure, prophylactically on a daily basis if desired, or therapeutically at the start of early viral symptoms.

Another object of the invention is to provide compositions of anti-inflammatory phytochemical extracts and nutraceuticals, already established to be safe for consumption that can be taken electively before anticipated viral exposure, prophylactically on a daily basis if desired, or therapeutically at the start of early viral symptoms.

Another object of the invention is to prevent or limit development of virally induced secondary bacterial infection/inflammation, or vice versa, especially in the lungs.

Another object of the invention is to provide steroid sparing or non-steroidal compositions and methods for treating inflammation in humans and animals.

An overall object of the invention is to help flatten the viral epidemic curve of yearly influenza, and that of any new viral epidemic.

Other features and advantages of the present invention will become apparent to those of skill in the art to consideration of the ensuing description and the appended claims.

Accordingly, the invention features a composition for preventing and for treating and reducing infections and tissue inflammation caused by bacteria and viruses in humans and animals, the composition including effective amounts of the following: vitamin C, vitamin E, a source of zinc, a source of taurine, one or more anti-infectious phytochemicals, one or more anti-inflammatory nutraceutical compounds, and one or more anti-inflammatory phytochemicals.

In another aspect, the invention can feature the one or more anti-infectious phytochemicals being or including: fulvic acid; Radix bupleuri extract; Echinacea purpura extract; sage extract; pomegranate extract; Uncaria tomentosa extract; Sambucus nigra extract; or Sambucus canadensis extract.

In another aspect, the invention can feature the one or more anti-inflammatory nutraceutical compounds being or including: an omega-3 fatty acid; eicosapentanoic acid (EPA); docosahexanoic acid (DHA); or alpha-lipoic acid.

In another aspect, the invention can feature the one or more anti-inflammatory phytochemicals being or including: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

In another aspect, the invention can feature the one or more anti-infectious phytochemicals including two or more of the following: fulvic acid; Radix bupleuri extract; Echinacea purpura extract; sage extract; Uncaria tomentosa extract; Sambucus nigra extract; Sambucus canadensis extract; or pomegranate extract. The one or more anti-inflammatory nutraceutical compounds include two or more of the following: an omega-3 fatty acid; eicosapentanoic acid (EPA); docosahexanoic acid (DHA); or alpha-lipoic acid. The one or more anti-inflammatory phytochemicals includes two or more of the following: curcumin extract or another source of piperine; green tea extracts; Boswellia serrata extract or another source of 3-acetyl-11-keto-beta-boswellic acid (AKBA); resveratrol; Commiphora myrrha extract; Uncaria tomentosa extract; or Pinus maritima bark extract.

In another aspect, the invention can feature the source of zinc being or including: elemental zinc; zinc methionine; zinc pyrithione; zinc citrate; zinc gluconate; zinc picolinate; zinc sulfate; zinc acetate; zinc orotate; or a combination of two or more of the foregoing.

In another aspect, the invention can feature the composition being incorporated in a tablet, a powder, a pill, a capsule, a nasal spray, an inhalable form, a suspension, a topically applied form, or a liquid for administration to a human or other animal via oral, parenteral, inhalation, topical application, or other administration routes.

The invention also features a composition for preventing and for treating and reducing infections and tissue inflammation caused by bacteria and viruses in humans and animals, the composition including effective amounts of the following: vitamin C; a second vitamin; a source of zinc; a source of taurine; one or more anti-infectious phytochemicals; one or more anti-inflammatory nutraceutical compounds; and one or more anti-inflammatory phytochemicals. The one or more anti-infectious phytochemicals is or are selected from among: fulvic acid; Radix bupleuri extract; Echinacea purpura extract; sage extract; Uncaria tomentosa extract; Sambucus nigra extract; Sambucus canadensis extract; or pomegranate extract. The one or more anti-inflammatory phytochemicals is or are selected from among: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

In another aspect, the invention can feature the second vitamin being or including vitamin E.

In another aspect, the invention can feature the source of zinc being selected from among: elemental zinc; zinc methionine; zinc pyrithione; zinc citrate; zinc gluconate; zinc picolinate; zinc sulfate; zinc acetate; zinc orotate; or a combination of two or more of the foregoing.

In another aspect, the invention can feature the one or more anti-inflammatory nutraceutical compounds being selected from among: an omega-3 fatty acid; eicosapentanoic acid (EPA); docosahexanoic acid (DHA); or alpha-lipoic acid.

The invention also features a composition for preventing and for treating and reducing infections and tissue inflammation caused by bacteria and viruses in humans and animals, the composition including effective amounts of the following: vitamin C; vitamin E; a source of zinc; a source of taurine; one or more anti-infectious phytochemicals; one or more anti-inflammatory nutraceutical compounds; and one or more anti-inflammatory phytochemicals. The source of zinc is selected from among: elemental zinc; zinc methionine; zinc pyrithione; zinc citrate; zinc gluconate; zinc picolinate; zinc sulfate; zinc acetate; zinc orotate; or a combination of two or more of the foregoing. The one or more anti-inflammatory nutraceutical compounds is selected from among: an omega-3 fatty acid; eicosapentanoic acid (EPA); docosahexanoic acid (DHA); or alpha-lipoic acid.

In another aspect, the invention can feature the one or more anti-infectious phytochemicals being selected from among: fulvic acid; Radix bupleuri extract; Echinacea purpura extract; sage extract; Uncaria tomentosa extract; Sambucus nigra extract; Sambucus canadensis extract; or pomegranate extract.

In another aspect, the invention can feature the one or more anti-inflammatory phytochemicals being selected from among: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol. The invention also features a composition for preventing and for treating and reducing infections and tissue inflammation caused by bacteria and viruses in humans and animals, the composition including effective amounts of the following: vitamin C; vitamin E; a source of zinc; a source of taurine; one or more anti-infectious phytochemicals; one or more anti-inflammatory nutraceutical compounds; and one or more anti-inflammatory phytochemicals. The source of zinc is selected from among: elemental zinc; zinc methionine; zinc pyrithione; zinc citrate; zinc gluconate; zinc picolinate; zinc sulfate; zinc acetate; zinc orotate; or a combination of two or more of the foregoing. The one or more anti-infectious phytochemicals is selected from among: fulvic acid; Radix bupleuri extract; Echinacea purpura extract; sage extract; Uncaria tomentosa extract; Sambucus nigra extract; Sambucus canadensis extract; or pomegranate extract. The one or more anti-inflammatory nutraceutical compounds is selected from among: an omega-3 fatty acid; eicosapentanoic acid (EPA); docosahexanoic acid (DHA); or alpha-lipoic acid. The one or more anti-inflammatory phytochemicals is selected from among: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

The invention also features an antiviral and antibacterial composition for preventing and for treating and reducing infections in humans and other animals caused by bacteria and viruses, the composition including effective amounts of the following: vitamin C, vitamin E, a source of zinc, and one or more anti-infectious phytochemicals.

The invention also features an anti-inflammatory composition for preventing and for treating and reducing tissue inflammation in humans and animals, the composition including effective amounts of the following: a source of taurine, one or more anti-inflammatory nutraceutical compounds, and one or more anti-inflammatory phytochemicals.

A method of the invention can be used for preventing and for treating and reducing infections and tissue inflammation caused by bacteria and viruses in humans and animals. The method includes the steps of: (a) preparing a composition that includes effective amounts of the following: vitamin C, vitamin E, a source of zinc, a source of taurine, one or more anti-infectious phytochemicals, one or more anti-inflammatory nutraceutical compounds, and one or more anti-inflammatory phytochemicals; (b) providing the composition in a tablet, a powder, a pill, a capsule, a nasal spray, an inhalable form, a suspension, a topically applied form, or a liquid form to be administered to a human or other animal via oral, parenteral, inhalation, topical application, or other administration routes; and (c) administering the composition to the human or other animal periodically.

Another method of the invention can include the one or more anti-infectious phytochemicals being or including: fulvic acid; Radix bupleuri extract; Echinacea purpura extract; sage extract; pomegranate extract; Uncaria tomentosa extract; Sambucus nigra extract; or Sambucus canadensis extract.

Another method of the invention can include the one or more anti-inflammatory nutraceutical compounds being or including: an omega-3 fatty acid; eicosapentanoic acid (EPA); docosahexanoic acid (DHA); or alpha-lipoic acid.

Another method of the invention can include the one or more anti-inflammatory phytochemicals being or including: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

Another method of the invention can include the source of zinc being or including: elemental zinc; zinc methionine; zinc pyrithione; zinc citrate; zinc gluconate; zinc picolinate; zinc sulfate; zinc acetate; zinc orotate; or a combination of two or more of the foregoing.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The present invention is best understood by reference to the description set forth herein. Embodiments of the invention are discussed below; however, those skilled in the art will readily appreciate that the detailed description given herein is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The invention provides compositions containing phytochemical extracts and nutraceuticals to fortify the immune systems of humans and animals in a way that prevents a wide spectrum of known or new strains of viruses and bacteria from starting a new infection in the host and that also permits nascent viral or bacterial infection, once established, to be treated and defeated before the infection can cause serious and/or widespread systemic symptoms and effects, or to minimize such symptoms to a life non-threatening level. The invention provides compositions containing phytochemical extracts and nutraceuticals that prevent or limit inflammation, especially in the lungs, in which virally or bacterially induced inflammation causes arteriolar and capillary leakage and resultant pulmonary edema, excess mucus secretion, possible severe bronchoconstriction and, in the worst case, acute respiratory distress syndrome (ARDS) with airway collapse, any of which phenomena can compromise pulmonary function to the point of fatal respiratory failure. The invention provides compositions containing anti-infective (i.e., antiviral and antibacterial) phytochemical extracts and nutraceuticals, already established to be safe for consumption that can be taken electively before anticipated viral exposure, prophylactically on a daily basis if desired, or therapeutically at the start of early viral symptoms. The invention provides compositions containing anti-inflammatory phytochemical extracts and nutraceuticals, already established to be safe for consumption that can be taken electively before anticipated viral exposure, prophylactically on a daily basis if desired, or therapeutically at the start of early viral symptoms. The invention provides compositions and methods that prevent or limit development of virally induced secondary bacterial infection/inflammation, or vice versa, especially in the lungs. The invention provides novel and unique combinations of vitamins, minerals and herbal/organic supplements (i.e., phytochemicals and nutraceuticals) in one formulary forming one or more compositions and methods for the purpose of fortifying the innate (cellular) and adaptive (hormonal) components of the immune system of a human or an animal against bacterial and viral infection, and their propagation, transmission and effects. The invention provides steroid sparing or non-steroidal compositions and methods for treating inflammation in humans and animals.

As used herein, the word “nutraceutical” refers to a food item, or a part of a food item, that offers medical health benefits, including prevention and/or treatment of disease. More particularly, a nutraceutical is a material that is: (a) a dietary supplement containing a nutritive bioactive compound; or (b) a biologically active material (processed or unprocessed) derived from a vitamin, mineral, plant, fungus, or animal, or a portion thereof, where the precise composition of the biologically active processed or unprocessed material may be undetermined.

As used herein, the word “phytochemical” refers to non-nutritive organic components of plants that are thought to promote human health. Unlike the traditional nutrients (e.g., protein, fat, carbohydrates, vitamins, and minerals), phytochemicals are not essential for life, but have a wide range of therapeutic applications. Phytochemicals, within the parent plant, help it resist microbial attack by fungi, bacteria, and viruses.

Examples of a biologically active processed material may include a finely chopped, powdered, pureed, or cooked material derived from plant, animal, or fungus tissue, or an extract of plant, animal, or fungus tissue, or combinations of the foregoing. These agents are useful for accelerating improved immune function, and they include extracts from various plants and mixtures thereof.

Where specific reference is made herein to a particular species of plant, one or more other species of the same genus may be substituted for the identified species when the related species is known or determined to be safe.

The compositions described herein include two or more natural, herbal or other organic anti-inflammatory substances for the suppression of tissue inflammation, especially in the lungs. The formulary thereby provides multimodal nutraceutical and phytochemical protection against acquisition of infection and against deleterious effects of inflammation in the lungs. The formulary is available in forms that permit administration to a human or other animal via oral, parenteral, inhalation, topical application, or other administration routes.

In one embodiment that has both anti-infectious (i.e., antiviral and/or antibacterial) properties and anti-inflammatory properties, the composition includes vitamin C, vitamin E, a source of zinc, a source of taurine, one or more anti-infectious phytochemicals, one or more anti-inflammatory nutraceutical compounds, and one or more anti-inflammatory phytochemicals.

In another embodiment that has both anti-infectious (i.e., antiviral and/or antibacterial) properties and anti-inflammatory properties, the composition includes: vitamin C, a second vitamin, a source of zinc, a source of taurine, one or more anti-infectious phytochemicals, one or more anti-inflammatory nutraceutical compounds, and one or more anti-inflammatory phytochemicals.

In another embodiment that has anti-infectious (i.e., antiviral and/or antibacterial) properties, the composition includes: vitamin C, vitamin E, a source of zinc, and one or more anti-infectious phytochemicals.

In another embodiment that has anti-inflammatory properties, the composition includes: a source of taurine, one or more anti-inflammatory nutraceutical compounds, and one or more anti-inflammatory phytochemicals.

In the embodiments described above and others described herein, the one or more anti-infectious phytochemicals included in the composition are selected from among: fulvic acid; Radix bupleuri extract; Echinacea purpura extract; sage extract; pomegranate extract; cat's claw (Uncaria tomentosa) extract; Black Elderberry (Sambucus nigra) extract; or American Elderberry (Sambucus canadensis) extract.

In the embodiments described above and others described herein, the one or more anti-inflammatory nutraceutical compounds included in the composition are selected from among: an omega-3 fatty acid; eicosapentanoic acid (EPA); docosahexanoic acid (DHA); or alpha-lipoic acid.

In the embodiments described above and others described herein, the one or more anti-inflammatory phytochemicals included in the composition are selected from among: Indian frankincense (Boswellia serrata) extract or another source of 3-acetyl-11-keto-beta-boswellic acid (AKBA); Maritime Pine (Pinus maritima) bark extract; cat's claw (Uncaria tomentosa) extract; myrrh (Commiphora myrrha) extract; resveratrol; curcumin extract or another source of piperine; green tea extract; or cannabidiol (CBD).

In the embodiments described above and others described herein that contain CBD, other cannabinoids may be substituted for CBD or used in combination with it.

The composition can include the following ranges of ingredients:
(a) 5 mg to 50 mg of a source of zinc
(b) 10 mg to 2,000 mg of vitamin C
(c) 5 mg to 1,000 mg of vitamin E
(d) five or more of the following wide-spectrum anti-infectious phytochemicals:
   5 micrograms to 2,000 mg of extracted fulvic acid
   5 micrograms to 1,000 mg of Radix bupleuri extract
   5 micrograms to 1,000 mg of Uncaria tomentosa extract
   5 micrograms to 1,750 mg of pomegranate extract
   5 micrograms to 2,000 mg of Black Elderberry (Sambucus nigra) extract, American Elderberry (Sambucus canadensis) extract, or a combination of the two
   5 micrograms to 3,200 mg of sage (Salvia officinalis) extract
   10 micrograms to 1,500 mg of Echinacea purpura extract
(e) two or more of the following anti-inflammatory nutraceuticals:
   5 micrograms to 1,000 mg of the omega-3 fatty acids, eicosapentanoic acid (EPA) and docosahexanoic acid (DHA)
   5 micrograms to 1,200 mg of alpha-lipoic acid
(f) 5 mg to 10,000 mg or more of a source of taurine
(g) five or more of the following anti-inflammatory phytochemicals:
   5 micrograms to 1,800 mg Boswellia serrata extract, containing its most anti-inflammatory moiety, 3-acetyl-11-keto-β-boswellic acid (AKBA)
   5 micrograms to 750 mg of Maritime Pine (Pinus maritima) bark extract
   5 micrograms to 400 mg of Uncaria tomentosa extract
   5 micrograms to 800 mg of Commiphora myrrha extract
   5 micrograms to 500 mg of resveratrol
   5 micrograms to 1,200 mg of curcumin extract with piperine
   5 micrograms to 900 mg of green tea extract
   5 micrograms to 680 mg of cannabidiol (CBD)

The following examples of exemplary embodiments of the composition are provided:

### EXAMPLE 1

In an exemplary embodiment, the composition contains:
(a) 25 mg of a source of zinc
(b) 1,000 mg of vitamin C
(c) 200 mg of vitamin E
(d) two or more of the following wide-spectrum anti-infectious phytochemicals:
   1,000 mg of extracted fulvic acid
   400 mg of Radix bupleuri extract
   400 mg of Uncaria tomentosa extract
   400 mg of pomegranate extract
   200 mg of Black Elderberry (Sambucus nigra) extract, American Elderberry (Sambucus canadensis) extract, or a combination of the two
   800 mg of sage (Salvia officinalis) extract
   800 mg of Echinacea purpura extract
(e) two or more of the following anti-inflammatory nutraceuticals:
   1,000 mg of the omega-3 fatty acids, eicosapentanoic acid (EPA) and docosahexanoic acid (DHA)
   200 mg of alpha-lipoic acid
(f) 250 mg or more of a source of taurine two or more of the following anti-inflammatory phytochemicals:
   800 mg Boswellia serrata extract, containing its most anti-inflammatory moiety, 3-acetyl-11-keto-β-boswellic acid (AKBA)
   150 mg of Maritime Pine (Pinus maritima) bark extract
   400 mg of Uncaria tomentosa extract
   400 mg of Commiphora myrrha extract
   200 mg of resveratrol
   400 mg of curcumin extract with piperine
   300 mg of green tea extract
   50 mg of cannabidiol (CBD)

### EXAMPLE 2

In another exemplary embodiment, the composition contains:
(a) 25 mg of a source of zinc
(b) 1,000 mg of vitamin C
(c) 200 mg of vitamin E
(d) two or more of the following wide-spectrum anti-infectious phytochemicals:
   1,000 mg of extracted fulvic acid
   400 mg of Radix bupleuri extract
   400 mg of Uncaria tomentosa extract
   400 mg of pomegranate extract
   200 mg of Black Elderberry (Sambucus nigra) extract, American Elderberry (Sambucus canadensis) extract, or a combination of the two
   800 mg of sage (Salvia officinalis) extract
   800 mg of Echinacea purpura extract
(e) two or more of the following anti-inflammatory nutraceuticals:
   1,000 mg of the omega-3 fatty acids, eicosapentanoic acid (EPA) and docosahexanoic acid (DHA)
   200 mg of alpha-lipoic acid
(f) 250 mg or more of a source of taurine
(g) two or more of the following anti-inflammatory phytochemicals:
   800 mg Boswellia serrata extract, containing its most anti-inflammatory moiety, 3-acetyl-11-keto-β-boswellic acid (AKBA)
   150 mg of Maritime Pine (Pinus maritima) bark extract
   400 mg of Uncaria tomentosa extract
   400 mg of Commiphora myrrha extract
   200 mg of resveratrol
   400 mg of curcumin extract with piperine
   300 mg of green tea extract
   50 mg of cannabidiol (CBD)

In the embodiments described above and others described herein, the source of zinc can be included in the composition as elemental zinc, zinc methionine, zinc pyrithione, zinc citrate, zinc gluconate, zinc picolinate, zinc sulfate, zinc acetate, zinc orotate, or a combination of two or more of the foregoing.

In the embodiments described above and others described herein, the second vitamin included in the composition can be selected from among one or more of vitamins E, A, D, K, niacin, riboflavin, thiamine, B₆, B₁₂, pantothenic acid, biotin, and folate, or a combination of two or more of the foregoing.

In the embodiments described above and others described herein, the source of taurine can be included in the composition as taurine, a taurine-containing compound, an extract of a functional food containing taurine or a taurine-containing compound, or a combination of two or more of the foregoing. A taurine-related compound may also be substituted for the source of taurine in some embodiments.

Any of the listed wide-spectrum anti-infectious nutraceuticals and phytochemicals permutated with any of the listed anti-inflammatory nutraceuticals and phytochemicals will confer five levels of anti-infectious protection, including white blood cell fortification and activation, virucidal-bactericidal activity, enhancement of antibody production, anti-oxidant activity, and anti-inflammatory activity. The oral formulary, capsule or other, may be enteric-coated to prevent breakdown by gastric acid and permit delivery of unaltered nutraceuticals and phytochemicals to the duodenum for absorption.

The compositions described above can be made in any of several various forms including, without limitation, as a tablet, a powder, a pill, a capsule, a nasal spray, an inhalable form, a suspension, a topically applied form, or a liquid form to be administered to a human or other animal via oral, parenteral, inhalation, topical application, or other administration routes. A topically applied form can be a cream, a lotion, a salve, an ointment, a gel, a paste, a topical solution, a tincture, a foam, or any other form capable of topical application to skin of a human or other animal. In some embodiments, the composition may be incorporated in a transdermal patch for application to the skin.

The compositions and methods described herein may also be effective at preventing and treating certain fungal, protozoal, and other microbial infections in humans and animals.

As referenced elsewhere herein, the invention also relates to a method for preventing and for treating and reducing infections and tissue inflammation caused by bacteria and viruses in humans and animals. The method includes the step of preparing a composition that includes effective amounts of the following: vitamin C, vitamin E, a source of zinc, a source of taurine, one or more anti-infectious phytochemicals, one or more anti-inflammatory nutraceutical compounds, and one or more anti-inflammatory phytochemicals. The source of zinc, source of taurine, one or more anti-infectious phytochemicals, one or more anti-inflammatory nutraceutical compounds, and one or more anti-inflammatory phytochemicals can be selected from among any of the compounds, extracts, and substances described herein for each of those categories. The method also includes the step of providing the composition in a tablet, a powder, a pill, a capsule, a nasal spray, an inhalable form, a suspension, a topically applied form, or a liquid form to be administered to a human or other animal via oral, parenteral, inhalation, topical application, or other administration routes. The method also includes the step of administering the composition to the human or other animal periodically. For example, the composition may be administered daily, twice daily, every four hours, every six hours, every other day, weekly, or at another appropriate interval. The exact interval may vary depending on factors related to the formula of the composition being administered, the purpose (treatment versus prophylaxis), the disease or condition being treated or prevented, the dosage, and the condition of the human or animal to whom or which the composition is being administered.

As referenced elsewhere herein, the invention also relates to a method for preventing and for treating and reducing infections caused by bacteria and viruses in humans and animals. The method includes the step of preparing a composition that includes effective amounts of the following: vitamin C, vitamin E, a source of zinc, and one or more anti-infectious phytochemicals. The source of zinc and one or more anti-infectious phytochemicals can be selected from among any of the compounds, extracts, and substances described herein for each of those categories. The method also includes the step of providing the composition in a tablet, a powder, a pill, a capsule, a nasal spray, an inhalable form, a suspension, a topically applied form, or a liquid form to be administered to a human or other animal via oral, parenteral, inhalation, topical application, or other administration routes. The method also includes the step of administering the composition to the human or other animal periodically. For example, the composition may be administered daily, twice daily, every four hours, every six hours, every other day, weekly, or at another appropriate interval. The exact interval may vary depending on factors related to the formula of the composition being administered, the purpose (treatment versus prophylaxis), the disease or condition being treated or prevented, the dosage, and the condition of the human or animal to whom or which the composition is being administered.

As referenced elsewhere herein, the invention also relates to a method for preventing and for treating and reducing infections and tissue inflammation caused by bacteria and viruses in humans and animals. The method includes the step of preparing a composition that includes effective amounts of the following: a source of taurine, one or more anti-inflammatory nutraceutical compounds, and one or more anti-inflammatory phytochemicals. The source of taurine, one or more anti-inflammatory nutraceutical compounds, and one or more anti-inflammatory phytochemicals can be selected from among any of the compounds, extracts, and substances described herein for each of those categories. The method also includes the step of providing the composition in a tablet, a powder, a pill, a capsule, a nasal spray, an inhalable form, a suspension, a topically applied form, or a liquid form to be administered to a human or other animal via oral, parenteral, inhalation, topical application, or other administration routes. The method also includes the step of administering the composition to the human or other animal periodically. For example, the composition may be administered daily, twice daily, every four hours, every six hours, every other day, weekly, or at another appropriate interval. The exact interval may vary depending on factors related to the formula of the composition being administered, the purpose (treatment versus prophylaxis), the disease or condition being treated or prevented, the dosage, and the condition of the human or animal to whom or which the composition is being administered.

### Other Embodiments

It is to be understood that while the invention has been described in conjunction with the detailed description thereof, the foregoing description is intended to illustrate and not limit the scope of the invention, which is defined by the scope of the appended claims. Other embodiments, aspects, advantages, and modifications are within the scope of the following claims.

What is claimed is:

1. A composition for preventing and for treating and reducing infections and tissue inflammation caused by bacteria and viruses in humans and animals, the composition comprising effective amounts of the following:
vitamin C;
vitamin E;
a source of zinc;
a source of taurine;
one or more anti-infectious phytochemicals;
one or more anti-inflammatory nutraceutical compounds; and
one or more anti-inflammatory phytochemicals.

2. The composition of claim 1, wherein the one or more anti-infectious phytochemicals comprise: fulvic acid; Radix bupleuri extract; Echinacea purpura extract; sage extract; pomegranate extract; Uncaria tomentosa extract; Sambucus nigra extract; or Sambucus canadensis extract.

3. The composition of claim 1, wherein the one or more anti-inflammatory nutraceutical compounds comprise: an omega-3 fatty acid; eicosapentanoic acid (EPA);
docosahexanoic acid (DHA); or alpha-lipoic acid.

4. The composition of claim 1, wherein the one or more anti-inflammatory phytochemicals comprise: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

5. The composition of claim 1, wherein the one or more anti-infectious phytochemicals comprises two or more of the following: fulvic acid; Radix bupleuri extract; Echinacea purpura extract; sage extract; Uncaria tomentosa extract; Sambucus nigra extract; Sambucus canadensis extract; or pomegranate extract;
wherein the one or more anti-inflammatory nutraceutical compounds comprises two or more of the following: an omega-3 fatty acid; eicosapentanoic acid (EPA); docosahexanoic acid (DHA); or alpha-lipoic acid; and
wherein the one or more anti-inflammatory phytochemicals comprises two or more of the following: curcumin extract or another source of piperine; green tea extracts; Boswellia serrata extract or another source of 3-acetyl-11-keto-beta-boswellic acid (AKBA); resveratrol; Commiphora myrrha extract; Uncaria tomentosa extract; or Pinus maritima bark extract.

6. The composition of claim 1, wherein the source of zinc comprises: elemental zinc; zinc methionine; zinc pyrithione; zinc citrate; zinc gluconate; zinc picolinate; zinc sulfate; zinc acetate; zinc orotate; or a combination of two or more of the foregoing.

7. The composition of claim 1, wherein the composition is incorporated in a tablet, a powder, a pill, a capsule, a nasal spray, an inhalable form, a suspension, a topically applied form, or a liquid for administration to a human or other animal via oral, parenteral, inhalation, topical application, or other administration routes.

8. A composition for preventing and for treating and reducing infections and tissue inflammation caused by bacteria and viruses in humans and animals, the composition comprising effective amounts of the following:
vitamin C;
a second vitamin;
a source of zinc;
a source of taurine;
one or more anti-infectious phytochemicals comprising: fulvic acid; Radix bupleuri extract; Echinacea purpura extract; sage extract; Uncaria tomentosa extract; Sambucus nigra extract; Sambucus canadensis extract; or pomegranate extract;
one or more anti-inflammatory nutraceutical compounds; and
one or more anti-inflammatory phytochemicals comprising: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

9. The composition of claim 8, wherein the second vitamin comprises vitamin E.

10. The composition of claim 8, wherein the source of zinc comprises: elemental zinc;
zinc methionine; zinc pyrithione; zinc citrate; zinc gluconate; zinc picolinate; zinc sulfate; zinc acetate; zinc orotate; or a combination of two or more of the foregoing.

11. The composition of claim 8, wherein the one or more anti-inflammatory nutraceutical compounds comprises: an omega-3 fatty acid; eicosapentanoic acid (EPA);
docosahexanoic acid (DHA); or alpha-lipoic acid.

12. A composition for preventing and for treating and reducing infections and tissue inflammation caused by bacteria and viruses in humans and animals, the composition comprising effective amounts of the following:
vitamin C;
vitamin E;
a source of zinc comprising: elemental zinc; zinc methionine; zinc pyrithione; zinc citrate; zinc gluconate; zinc picolinate; zinc sulfate; zinc acetate; zinc orotate; or a combination of two or more of the foregoing;
a source of taurine;
one or more anti-infectious phytochemicals;
one or more anti-inflammatory nutraceutical compounds comprising: an omega-3 fatty acid; eicosapentanoic acid (EPA); docosahexanoic acid (DHA); or alpha-lipoic acid; and
one or more anti-inflammatory phytochemicals.

13. The composition of claim 12, wherein the one or more anti-infectious phytochemicals comprise: fulvic acid; Radix bupleuri extract; Echinacea purpura extract; sage extract; Uncaria tomentosa extract; Sambucus nigra extract; Sambucus canadensis extract; or pomegranate extract.

14. The composition of claim 12, wherein the one or more anti-inflammatory phytochemicals comprise: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

15. A composition for preventing and for treating and reducing infections and tissue inflammation caused by bacteria and viruses in humans and animals, the composition comprising effective amounts of the following:
vitamin C;
vitamin E;
a source of zinc comprising: elemental zinc; zinc methionine; zinc pyrithione; zinc citrate; zinc gluconate; zinc picolinate; zinc sulfate; zinc acetate; zinc orotate; or a combination of two or more of the foregoing;
a source of taurine;
one or more anti-infectious phytochemicals comprising: fulvic acid; Radix bupleuri extract; Echinacea purpura extract; sage extract; Uncaria tomentosa extract; Sambucus nigra extract; Sambucus canadensis extract; or pomegranate extract;
one or more anti-inflammatory nutraceutical compounds comprising: an omega-3 fatty acid; eicosapentanoic acid (EPA); docosahexanoic acid (DHA); or alpha-lipoic acid; and
one or more anti-inflammatory phytochemicals comprising: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

16. An antiviral and antibacterial composition for preventing and for treating and reducing infections in humans and other animals caused by bacteria and viruses, the composition comprising effective amounts of the following:
vitamin C;
vitamin E;
a source of zinc; and
one or more anti-infectious phytochemicals.

17. An anti-inflammatory composition for preventing and for treating and reducing tissue inflammation in humans and animals, the composition comprising effective amounts of the following:
a source of taurine;
one or more anti-inflammatory nutraceutical compounds; and
one or more anti-inflammatory phytochemicals.

18. A method for preventing and for treating and reducing infections and tissue inflammation caused by bacteria and viruses in humans and animals, the method comprising the steps of:
(a) preparing a composition comprising effective amounts of the following:
vitamin C;
vitamin E;
a source of zinc;
a source of taurine;
one or more anti-infectious phytochemicals;
one or more anti-inflammatory nutraceutical compounds; and
one or more anti-inflammatory phytochemicals;
(b) providing the composition in a tablet, a powder, a pill, a capsule, a nasal spray, an inhalable form, a suspension, a topically applied form, or a liquid form to be administered to a human or other animal via oral, parenteral, inhalation, topical application, or other administration routes; and
(c) administering the composition to the human or other animal periodically.

19. The method of claim 18, wherein the one or more anti-infectious phytochemicals comprise: fulvic acid; Radix bupleuri extract; Echinacea purpura extract; sage extract; pomegranate extract; Uncaria tomentosa extract; Sambucus nigra extract; or Sambucus canadensis extract.

20. The method of claim 18, wherein the one or more anti-inflammatory nutraceutical compounds comprise: an omega-3 fatty acid; eicosapentanoic acid (EPA); docosahexanoic acid (DHA); or alpha-lipoic acid.

21. The method of claim 18, wherein the one or more anti-inflammatory phytochemicals comprise: Boswellia serrata extract or another source of 3-acetyl-11-keto-beta-boswellic acid (AKBA); Pinus maritima bark extract; Uncaria tomentosa extract; Commiphora myrrha extract; resveratrol; curcumin extract or another source of piperine; green tea extract; or cannabidiol.

22. The method of claim 18, wherein the source of zinc comprises: elemental zinc; zinc methionine; zinc pyrithione; zinc citrate; zinc gluconate; zinc picolinate; zinc sulfate; zinc acetate; zinc orotate; or a combination of two or more of the foregoing.
